(19) United States
(12) Patent Application Publication
(10) Pub. No.: US 2023/0259820 A1
(43) Pub. Date: Aug. 17, 2023

(54) SMART SELECTION TO PRIORITIZE DATA COLLECTION AND ANNOTATION BASED ON CLINICAL METRICS

(71) Applicant: Siemens Healthcare GmbH, Erlangen (DE)

(72) Inventors: Noha El-Zehiry, Plainsboro, NJ (US); Gareth Funka-Lea, Princeton, NJ (US); Puneet Sharma, Princeton Junction, NJ (US)

(21) Appl. No.: 18/066,079

(22) Filed: Dec. 14, 2022

Related U.S. Application Data
(60) Provisional application No. 63/268,113, filed on Feb. 16, 2022.

(51) Int. Cl. G06N 20/00; G16H 50/50

(52) U.S. Cl. CPC G06N 20/00; G16H 50/50

(57) ABSTRACT

Systems and methods for smart selection of training data sets by using clinically driven application dependent evaluation metrics to assess the performance of deep learning models after deployment in the field. A machine trained model is deployed to a clinical environment. An evaluation metric is acquired that correlates with a clinical outcome for each instance of the machine trained model performing the task for a medical procedure. Data sets are flagged that are challenging for the machine trained model based on the evaluation metrics. The flagged data sets are prioritized during retraining of the machine trained model.

### CROSS REFERENCE TO RELATED APPLICATIONS

This patent document claims the benefit of the filing date under 35 U.S.C. § 119(e) of U.S. Provisional Pat. Application Ser. No. 63/268,113 filed on Feb. 16, 2022, which is hereby incorporated in its entirety by reference.

### FIELD

This disclosure relates to machine learning applications such as used in medical imaging.

### BACKGROUND

Artificial intelligence (AI) is a crucial component that is used for various medical procedures such as medical imaging applications and allows for precision medicine and an improved patient experience. The use of AI helps automate and standardize not only workflows but also complex diagnostics. AI is a computer-aided process for solving complex problems that are usually reserved for humans. Some examples are machine vision, pattern recognition, speech recognition, and knowledge-based decision-making.

Machine learning is a specific type of AI that uses models that are trained and improved by continuously inputting high volumes of data allowing the models to keep improving their performance expectations. Machine learning enables the model to adapt to new circumstances and to detect and extrapolate patterns. Deep (machine) learning is a type of machine learning that, for example, uses multilayer neural networks with multiple hidden layers between the input and output layers. These models / networks may identify relationships that may not have been recognized using traditional techniques.

In order to make accurate predictions or classifications, machine learning models leverage vast amounts of data. Machine learning models are configured using sample data, known as training data, in order to make these predictions or decisions by repeatedly inputting the training data and comparing the output of the model to an expected output. Through the processes of, for example, gradient descent and backpropagation, machine learning models are adjusted over and over until the model is capable of making predictions about new inputs with an acceptable level of precision. Once trained to this acceptable level, a model may be deployed for use in a clinical environment.

High-quality data is a key ingredient for continuously improving the results of the models. The strength of the models comes from the wealth of the data used to train such models. In other words, the model is only as good as the training data on which it “learns”. The variability in this training data is a key component in building effective deep learning models. In medical applications, one goal is to have a balanced population of training data that is collected for different genders, ages, from different acquisition sites, using different acquisition protocols, and potentially from different machine vendors so that the model “learns” from a wide variety of scenarios. In an example, current deep learning models may access and be trained on millions or billions or more of curated images, reports, and clinical and operational data. With the current wealth of information and millions of new data sets collected every day, it becomes more and more important to find a way to prioritize the data sets that should be integrated in the training pipeline for the deep learning models in order to efficiently train the deep learning models.

In addition, successful supervised training methods require accurate ground truth associated with each data entry. For example, when solving a segmentation problem, the ground truth boundary/segmentation must be drawn by an expert to train the respective machine learning model. This is a tedious task that requires a lot of time and resources. Given a limited number of resources for data preparation and annotation and given a relatively large amount of data, an issue becomes how to find subsets of the data that have the largest potential in improving the machine learning model to prioritize the data processing and/or assign the most difficult tasks to the annotators with the most expertise on the topic.

### SUMMARY

By way of introduction, the preferred embodiments described below include methods, systems, instructions, and computer readable media for identifying and prioritizing certain sets of data to be used during subsequent training of deep learning models.

In a first aspect, a method for smart selection of training data sets, the method comprising: machine training a model to perform a task; deploying the machine trained model to a clinical environment; acquiring an evaluation metric that correlates with a clinical outcome for each instance of the machine trained model performing the task for a medical procedure; and flagging data sets that are challenging for the machine trained model based on the clinical evaluation metrics.

In an embodiment, the method further includes prioritizing the flagged data sets during retraining of the machine trained model. Prioritizing the flagged data sets includes wherein the flagged data sets are given priority in at least one of data transfer, data anonymization, or preprocessing during retraining of the machine trained model. Flagging the data sets may include automatically assigning a priority score to the data sets and prioritizing the data sets may include pushing the data sets to an annotation queue according to their priority score. Prioritizing the flagged data sets may include assigning the flagged data sets to annotators such that expert annotators get the most difficult data sets to annotate. Prioritizing may include identifying sites in a federated learning setup that include the most challenging data sets.

In an embodiment, during machine training of the model, the model is tested using a testing metric that is different than the evaluation metric. The task of the model may include image segmentation of medical imaging data acquired using one of MRI, CT, X-ray, or Ultrasound.

The evaluation metric may include an assessment of how well an output of the machine trained model guided a clinician during the medical procedure subsequent to the performance of the task. Flagging may include flagging data sets that are the ten percent most challenging data sets.

In a second aspect, a system for smart data selection for training a deep learning network, comprising: a datastore configured to store a plurality of data sets, wherein each data set of the plurality of data sets is assigned an evaluation metric that correlates with a clinical outcome associated with a respective data set; the deep learning network configured to perform a task; and a processor configured to train the deep learning network using the plurality of data sets, the processor configured to prioritize data sets for use in the training based on the evaluation metric.

In an embodiment, during training of the deep learning network, the deep learning network is tested using a testing metric that is different than the evaluation metric. The evaluation metric may include an assessment of how well an output of the deep learning network guided a clinician during a medical procedure.

In an embodiment, the processor is configured to prioritize the data sets in at least one of data transfer, data anonymization, or preprocessing during training of the deep learning network. The processor may be configured to prioritize the data sets by assigning the data sets to annotators such that expert annotators get the most difficult data sets to annotate. The processor may be configured to assign the data sets a priority score based on the evaluation metric and prioritize the data sets by pushing the data sets to an annotation queue according to their priority score. The processor may be configured to only use the ten percent most challenging data sets based on the evaluation metric for subsequent training of the deep learning network.

In a third aspect, a method for smart data collection, the method comprising: performing a medical imaging procedure to generate medical imaging data; processing the medical imaging data using a machine learned network; computing an evaluation metric for the processed medical imaging data based on a clinical outcome related to the medical imaging procedure; determining, based on the evaluation metric, that the medical imaging data comprises a difficult data set; and prioritizing the medical imaging data for retraining of the machine learned network.

Prioritizing may include at least one of data transfer, data anonymization, or preprocessing of the medical imaging data during retraining of the machine learned network. Prioritizing the medical imaging data may include assigning the medical imaging data to annotators such that an expert annotator gets the medical imaging data to annotate.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The components and the figures are not necessarily to scale, emphasis instead being placed upon illustrating the principles of the embodiments. Moreover, in the figures, like reference numerals designate corresponding parts throughout the different views.

FIG. 1 depicts an embodiment of a system for training and deploying a model.

FIG. 2 depicts an example distribution of data sets.

FIG. 3 depicts an example workflow for smart selection of data sets for training a model according to an embodiment.

FIGS. 4A and 4B depict an example of the possible location of the ablation tags relative to the segmentation surface.

FIG. 5 depicts an example of a liver resection.

FIGS. 6A and 6B depict planning for an automatic liver resection.

FIG. 7 depicts an example system for smart selection of data sets for training a model according to an embodiment.

FIG. 8 depicts an example flowchart for smart selection of data sets for training a model according to an embodiment.

FIG. 9 depicts another example of a workflow for smart selection of data sets for training a model according to an embodiment.

### DETAILED DESCRIPTION

Embodiments described herein provide systems and methods for smart selection of training data sets by designing and using clinically driven application dependent evaluation metrics to assess the performance of deep learning models after deployment in the field. Data sets are flagged where the evaluation metric falls outside an acceptable range. This may indicate that the data sets are problematic and that the current model does not behave properly on them. For example, when the original training data set does not include enough information that resembles the test data sets, the model may perform poorly when presented with such data sets after deployment. Embodiments prioritize the preprocessing of the flagged data sets and/or their incorporation in training in the next development cycle based on the evaluation metric values.

Machine learning and deep learning models are capable of different types of learning such as supervised learning, unsupervised learning, and reinforcement learning. Supervised learning utilizes labeled / annotated datasets to categorize or make predictions which in most cases requires some kind of human intervention to label input data correctly. The data is known as training data and includes a set of training examples. Through iterative optimization of an objective function, supervised learning models learn a function that can be used to predict the output associated with new inputs. An optimal function allows the model to correctly determine the output for inputs that were not a part of the training data. A model that improves the accuracy of its outputs or predictions over time is said to have learned to perform that task. The following examples use supervised learning, although embodiments may be configured for other types of learning such as unsupervised or reinforcement learning depending on the network, task, and available training data.

Unsupervised learning doesn’t require labeled datasets, and instead, it detects patterns in the data, clustering them by any distinguishing characteristics. Unsupervised learning lacks individual target variables and instead have the goal of characterizing a data set in general. Unsupervised machine learning algorithms are often used to group (cluster) data sets, e.g., to identify relationships between individual data points (that may include of any number of attributes) and group them into clusters. Different algorithms or techniques may be used such as clustering. Clustering is the process of partitioning data into groups according to certain characteristics of data. Clustering splits data into groups of similar objects. Every group, called cluster, includes of members that are quite similar and members from the various clusters are different from each other. Reinforcement learning uses software agents and actions in an environment so as to maximize some notion of cumulative reward.

The deep learning models (also referred to as networks or neural networks) may include multiple layers of interconnected nodes, each building upon the previous layer to refine and optimize the prediction or categorization. This progression of computations through the network is called forward propagation. The input and output layers of a deep neural network are called visible layers. The input layer is where the deep learning model ingests the data for processing, and the output layer is where the final prediction or classification is made. The deep learning models use backpropagation and functions such as gradient descent, to calculate errors in predictions and then adjust the weights and biases of the function by moving backwards through the layers in an effort to train the model. Together, forward propagation and backpropagation allow the model to make predictions and correct for any errors accordingly. Over time, the model becomes gradually more accurate.

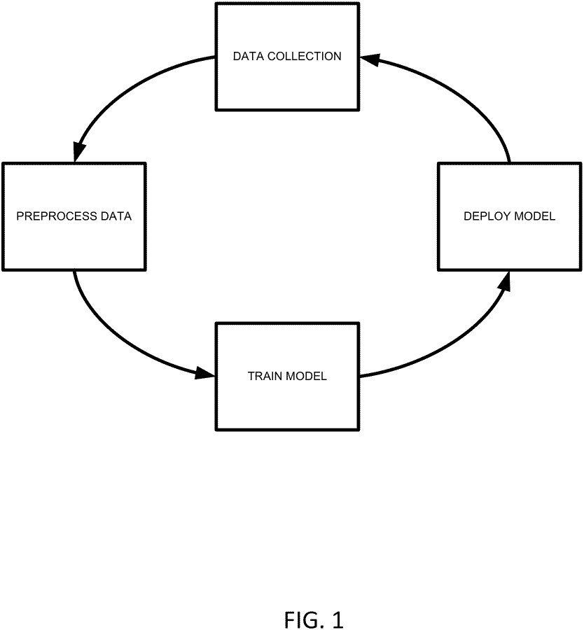

FIG. 1 depicts an example of the training and deployment cycle for typical deep learning models. Data is collected. The data is processed (annotated). The model is trained and then deployed. The performance of the model is typically tested at the training stage and prior to deployment. Additional data is collected and the process repeats. Each step requires resources and takes time to implement. In particular, the data collection step and processing step may be labor and time intensive. The training phase may also require extensive computing resources and time.

The training phase includes repeatedly inputting training data into the model and comparing the output to the expected (annotated) outcome. In a simple case, the model takes input variables (x) and an output variable (Y). The model learns the mapping function from the input to the output. Y = f(X). The goal is to approximate the mapping function so well that when input new input data (x) the model can predict the output variables (Y) for that data. Different tasks may be performed by the model including image processing and medical diagnosis among other tasks. In an example, models may be used to analyze and annotate medical images. For example, a model may be used in medical imaging (such as CT data, X-rays, or MRI scans) to look for patterns that indicate a particular disease or abnormality. This may help physicians or operators make quicker, more accurate diagnoses. Machine learning models may also help process image data so that users may more efficiently and accurately perform procedures. Machine learning models may be used to segment / contour / partition image data. Machine learning models may be used to clean or denoise image data. Machine learning or training of the model is heavily dependent on the type and quality of training data that is used.

The training data may be acquired at any point prior to inputting the training data into the model. In an example operation, the model is configured to segment an input image. The model inputs the training data and outputs a segmented image. The prediction is compared to, for example, a hand segmented image of the input data. A loss function may be used to identify the errors from the comparison. The loss function serves as a measurement of how far the current set of predictions are from the corresponding true values. Some examples of loss functions that may be used include Mean-Squared-Error, Root-Mean-Squared-Error, and Cross-entropy loss. Mean Squared Error loss, or MSE for short, is calculated as the average of the squared differences between the predicted and actual values. Root-Mean Squared Error is similarly calculated as the average of the root squared differences between the predicted and actual values. During training and over repeated iterations, the network attempts to minimize the loss function as the result of a lower error between the actual and the predicted values means the network has done a good job in learning. Different optimization algorithms may be used to minimize the loss function, such as, for example, gradient descent, Stochastic gradient descent, Batch gradient descent, Mini-Batch gradient descent, among others. The process of inputting, outputting, comparing, and adjusting is repeated for a predetermined number of iterations with the goal of minimizing the loss function. The model may be tested using one or more segmentation-based evaluation metrics such DICE, Jaccard, true positive rate, true negative rate, modified Hausdorff, volumetric similarity, or others. DICE, for example, is a measure of the comparison between two different images or sets of values. The Jaccard index (JAC) between two sets is defined as the intersection between them divided by their union. True Positive Rate (TPR), also called Sensitivity and Recall, measures the portion of positive voxels in the ground truth that are also identified as positive by the segmentation being evaluated. Analogously, True Negative Rate (TNR), also called specificity, measures the portion of negative voxels (background) in the ground truth segmentation that are also identified as negative by the segmentation being evaluated. Once trained to an acceptable level, the model may be deployed for use in a clinical setting.

Machine learned models need to be updated over time. Models may be initially trained on data sets that are not reflective of the clinical environment and thus may fail or perform poorly when first deployed. Available datasets only partially reflect the clinical situation for a particular medical condition. As an example, a dataset collected as part of a population study might have different characteristics that people who are referred to the hospital for treatment (higher incidence of a disease). Dataset bias occurs when the data used to build the model (the training data), has a different distribution than the data on which it should be applied (the test data). To assist in clinically relevant predictions, the test data must match the actual target population, rather than be a random subset of the same data pool as the training data. With such a mismatch, models that perform well during testing may poorly in real world scenarios.

In addition, when models are deployed, the performance typically falls off from the training / testing phase. This is because the model may be sensitive to changes in the real world, and user behavior keeps changing with time. Although all machine learning models decay, the speed of decay varies with time. This is mostly caused by data drift, concept drift, or both. Models typically do not keep working properly forever after deployment as the data or use may change. A model deployed and left to itself won’t be able to adapt to changes in data by itself, for example if operating procedures in a clinical setting change over time due to different patient populations, different operators use different equipment, new studies, technological advances in data acquisition etc. For a model to predict accurately, the data that it is making predictions on must have a similar distribution as the data on which the model was trained. Because data distributions can be expected to drift over time, deploying a model is not a one-time exercise but rather a continuous process. It is typical to retrain models on newer data as the newer data is acquired. Continuous training is an aspect of machine learning operations that automatically and continuously retrains machine learning models to adapt to changes in the data before it is redeployed. In an example, a model may be periodically updated, for example, daily, weekly, monthly, or as new data is acquired.

Retraining a model may lead to additional complexities or costs. Some of the complexities or costs that are associated with retraining a model include computational costs and labor cost. Training models may be very expensive and take a lot of time and computational resources. Manual labor may also be required to preprocess, clean, and annotate new training data. In addition, retraining may entail unnecessary costs because retraining a model doesn’t necessarily mean improved model performance if the “new” training data used is similar or redundant to the previously used training data.

Current solutions include a clinician reviewing the output of the model at the clinical site. The clinician manually selects data sets where the performance is not satisfactory and manually analyzes the cause to communicate the drawbacks to the development team. Alternatively, all of the collected data is transferred to the development team without any recommendation on data prioritization. This means that the development team has to use a lot of resources to preprocess and annotate all the transferred data set, or the development team randomly select a subset that might be redundant or already fairly represented in the training data set. All of these tasks are performed without knowing if the new data will improve the model or if the new data is redundant.

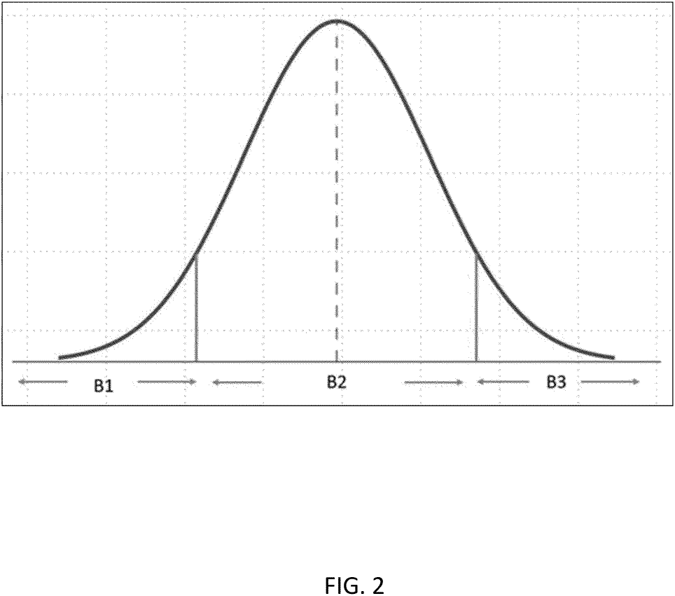

Because deep learning models learn from training examples, it is important that the test cases are drawn from a distribution that resembles the distribution of the training examples. Since it is almost impossible to cover every single variation that the model may be exposed to, it is always important to keep improving models by incorporating more variations after deployment. Theoretically speaking, it would be ideal if the distribution of the training data is uniform and equally covers all the possible variabilities in the data acquisition. Such variabilities include, but not limited to, age, gender, ethnicity, acquisition protocol, acquisition center, acquisition machine. Practically, it is infeasible to collect a perfect training data set that uniformly represents all these variations. Assuming the data collected for initial training is normally distributed as shown in FIG. 2. FIG. 2 depicts a bell curve of collected data. Other data distributions are possible and this example does not limit the application to normally distributed data. Most of the data samples would come from the middle part of the distribution, B2. Samples from both tails of the distribution, B1 and B3, are underrepresented in the training data set. To improve the performance of the model, adding data sets from B1 and B3 has a bigger impact than adding data sets from B2 that are already well represented in the training distribution.

In an example, Intracardiac Echocardiography (ICE) is used to acquire images of the cardiac structures (e.g., Left atrium and Pulmonary veins). These images are used to guide the cardiologists to ablate the heart tissue in the correct spots. This requires accurate definition of the boundaries of the different anatomies. Traditionally, a clinical application Specialist (CAS) examines each image frame and draws the boundaries of the existing anatomies. Deep learning models have provided an automatic segmentation solution. The model learns to provide a 3D segmentation of the anatomies from a sparse set of frames. In Left atrium ablation, these set of frames are conventionally acquired by inserting the catheter in the right atrium and clocking clockwise and counterclockwise with specific angles to obtain images of the left atrium, left atrial appendage and pulmonary veins. This acquisition protocol is the typical acquisition protocol that captures most of the data sets. However, occasionally, a cardiologist may decide to take different views that do not conform to the standard acquisition protocol. Since any model performance degrades when it is tested on a data set drawn from a distribution that is different from the training data set distribution, it becomes crucial to identify such data sets and use them to enrich the training data set. Conventionally, at the clinical site a clinician reviews the output of the model and manually selects data sets where the performance is not satisfactory, manually analyzes the cause, and communicates the drawbacks to the development team. Alternatively, and more often, the clinical site transfers all the collected data to the development team without any recommendation. Referring to FIG. 2, most of the data acquired by the conventional acquisition protocol falls in B2 and thus has minimal impact on improving the model.

Embodiments provided herein identify data sets (for example in bands B1 and B3 of FIG. 2) that deviate from the conventional data whether in the acquisition protocol, patient population, image quality and any other factor that would cause the data set to be underrepresented. Embodiments extend the training data set efficiently by adding more data that may be underrepresented in the original training distribution and giving lower priority to data sets that were already fairly represented and hence, considered redundant. Embodiments use an application dependent clinical evaluation metric that correlates with the clinical outcome to identify these data sets. An acceptable range is defined for the proposed evaluation metric. Data sets are identified where the evaluation metric falls outside the acceptable range. This indicates that these data sets are problematic, and the current algorithm does not behave properly on them. The original training data set does not include enough information that resembles the test data sets. The identified data sets are sorted in descending/ascending order based on the metric. The higher the deviation from the normal range, the more important the data set is. Embodiments prioritize the preprocessing of the data sets and their incorporation in training in the next development cycle based on the sorted metric values. Unlike the conventional evaluation metrics, such as DICE score for segmentation problems and confusion matrix for classification problems, the clinically designed metrics focus more on the impact of the results on the clinical application and thus the real-world effectiveness of the model.

Embodiments provide efficient deep learning models at lower costs. The ability to select the data sets that mostly enrich the training means that less time and cost are consumed on preprocessing and integrating redundant data sets that do not necessarily improve the model’s performance. Moreover, selecting nonredundant data sets introduces higher information content during the training and hence leads to better model performance. Embodiments reduce the cost and time of development and will provide more precise models. Embodiments provide smart selection for data. Smart selection refers to the process of selecting specific data sets that highly impact the performance of the models, as opposed to collecting random data sets. Therefore, the cost of data transfer, data storage, data annotation and preprocessing will be reduced. Smart selection also benefits the performance of the models because it selects the data with the highest information content, as opposed to redundant data sets. Such data sets impact the performance of the model the most. Embodiments further shorten the development cycle. Smart selection eliminates the need to wait for thousands of data sets to be transferred, preprocessed, and annotated to train a new model. Instead, only the data that has the highest potential of improving the model performance must be acquired. This reduces the time for the development cycles and allow models to be updated quickly to meet the customers’ needs.

Smart selection may also sort out the data based on how challenging it is to the algorithm. This enables the developers to assign the most challenging cases to the most expert annotators. Embodiments further may provide site optimization in a federated learning setup. Smart collection may identify data based on its value to the learning process. In a federated learning setup, this can be used to select the sites the provide the most valuable data.

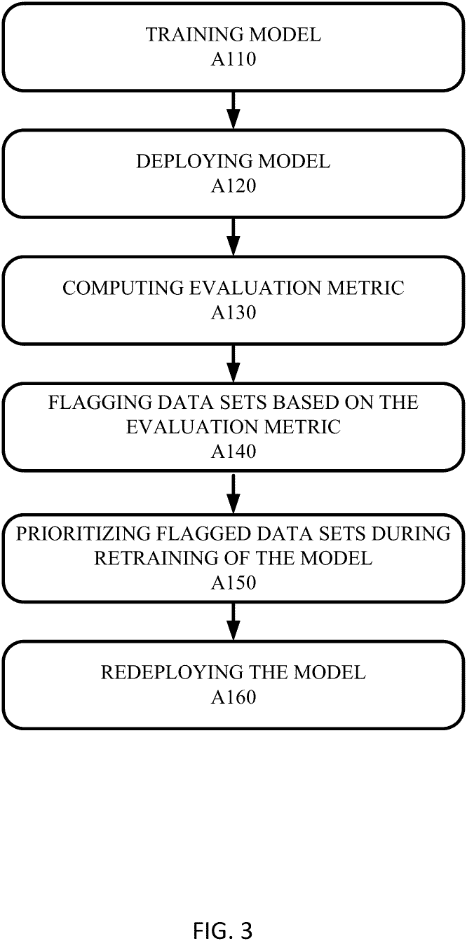

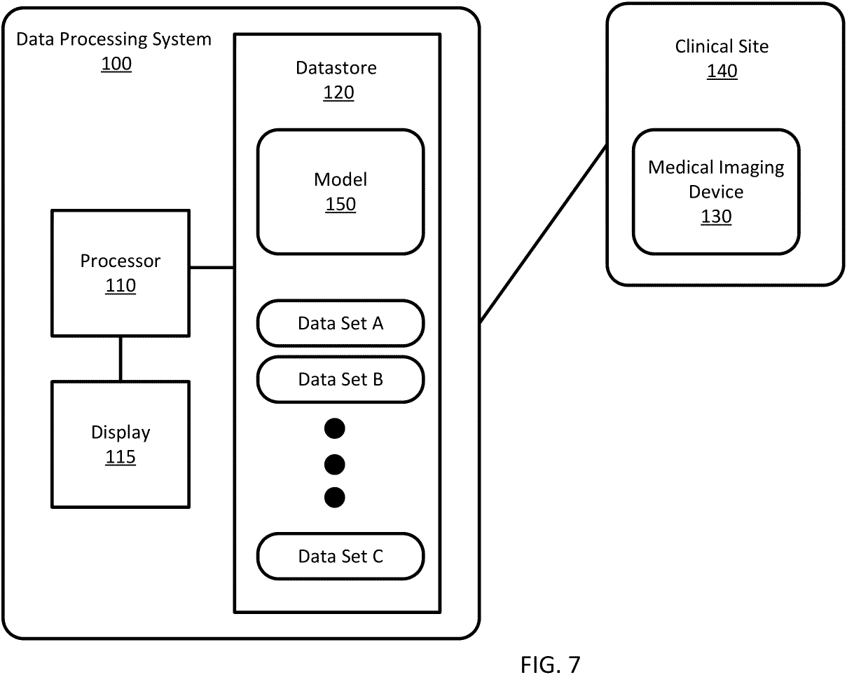

FIG. 3 depicts an example flowchart for smart selection of training data sets in order to provide efficient deep learning models. As presented in the following sections, the acts may be performed using any combination of the components indicated in FIG. 7, a processor, a datastore, a clinical site, or a combination thereof. Additional, different, or fewer acts may be provided. For example, additional information may be acquired about the user and used to estimate hair density and/or generate a new image. The acts are performed in the order shown or other orders. The acts may also be repeated. Certain acts may be skipped. The Acts A110 to A170 may be repeated multiple times. Certain acts, such as the deployment and acquisition of the evaluation metric may be performed multiple times in order to acquire additional sets of data. For example, the model may perform a hundred, a thousand, or more procedures prior to being retrained. The model used herein uses supervised training, but the model may also be configured to learn using unsupervised training or reinforcement learning.

At act A110, a model is trained to perform a task. Any trainable model may be used. In the examples provides below, the model may be a deep learning network configured to, for example, segment (contour / partition) an input image or volume. In an embodiment, the network is defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction based on an input image data with or without pre-processing. The features are learned to reconstruct lower-level features (i.e., features at a more abstract or compressed level). For example, features for reconstructing an image are learned. For a next unit, features for reconstructing the features of the previous unit are learned, providing more abstraction. Each node of the unit represents a feature. Different units are provided for learning different features.

Various units or layers may be used, such as convolutional, pooling (e.g., max pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. For example, the first unit provides features from the image, such as one node or feature being a line found in the image. The next unit combines lines, so that one of the nodes is a corner. The next unit may combine features (e.g., the corner and length of lines) from a previous unit so that the node provides a shape indication. For transposed convolution to reconstruct, the level of abstraction reverses. Each unit or layer reduces the level of abstraction or compression. Different types or configurations may be used for the model or network.

Training is the process of inputting sample / training data into the model and receiving an output. The output is compared with an annotated expected output. Based on a loss function that describes the difference between the output and the expected output, the network is adjusted. This process is repeated hundreds, thousands, or more times until the output of the network reaches an acceptable level. The loss function is a measurement of how good the model is in terms of predicting the expected outcome. Different loss functions may be used during training and/or for testing the model. Two segmentation loss functions that may be used include cross-entropy and DICE. Cross-entropy is used to measure the difference between two probability distributions. It is used as a similarity metric to tell how close one distribution of random events is to another and is used for both classification (in the more general sense) as well as segmentation. DICE is used to calculate the similarity between images and is similar to the Intersection-over-Union heuristic.

At act A120, the machine trained model is deployed to a clinical environment. The machine trained model may be used in any type of medical application, for example, single- or multi-modal (MRI, CT, Ultrasound, PET, SPECT) imaging. Different regions or organs may be imaged by these medical imaging devices. The machine learned model may input the raw data, an image, or a volume and segment the data into meaningful regions. From the segmentation, diagnostic measures and values may then be derived. The diagnostic measures and values may be used for diagnosis, planning, or other medical applications. In an embodiment, the machine learned model is trained and deployed for a specific task, e.g., segmentation of a particular organ. In other embodiments, the machine learned model may be trained generally for segmentation and deployed for different organs or regions depending on the procedure. In an embodiment, the machine trained model is configured to input Magnetic Resonance Imaging, Ultrasound, X-ray, and/or Computed Tomography data and output a segmented mask of a region. At act A130, the machine trained model performs the task for one or more procedures. The task may be performed automatically. During performance or after performance, information about the task performed may be collected, for example for use in indirectly or directly computing the evaluation metric.

At act A140, an evaluation metric is acquired or computed for the performance of the machine trained model that correlates with a clinical outcome for each of the one or more procedures. The evaluation metric is included with or assigned to the data set. The term data set refers to a collection of data that is used in a procedure and is relevant to the use of the machine learning model. The data set may be subsequently used to train or configure a machine learning model. A data set may include a single image, image data, multiple images, a volume, patient data, data describing the procedure etc. Certain models may have a single input, such as an image. Other models may have multiple inputs. Additional models may be used together and may be treated as a single system. In an example, a first model may take an input and generate an output. A second model may take this output as an input and generate a second output. The models may be trained / configured separately, or the models may be trained end to end.

The evaluation metric is specific to the task being performed and may require additional processing or evaluation. During training of a model, a cost function or loss function may be used to improve the model by comparing the outcome of the model to an expected outcome. The evaluation metric may be different than this cost function or loss function as the evaluation metric is correlated with a clinical outcome. The evaluation metric is thus not necessarily related to how well the model performed compared to manually annotated data, but rather how well the model assisted in the clinical setting. In an example, certain segmentation models may be more accurate but may hinder or lead to worse outcomes than other models. This may be due to operator preferences or assumptions, patient populations, or other differences between clinical settings and theory.

The evaluation metric may be quantified and ranked. For certain procedures a higher value may indicate a better performance, while for other procedures a higher value may indicate more issues. Different metrics and ranking / prioritization systems may be used for different procedures and different models. Two examples are described below for Atrial Fibrillation Planning using ICE Images and Liver Resection Surgery Planning from CT scans. These examples provide an overview of how an evaluation metric may be used to rank or score potential training data for subsequent training sessions. These examples are not limiting and may be extended to other procedures or may be altered. In an example, the evaluation metrics described herein are proposals and may be improved upon depending on the procedures and data collection processes.

Intracardiac Echocardiography (ICE) is used to acquire images of the cardiac structures (e.g., Left atrium and Pulmonary veins). These images are used to guide the cardiologists to ablate the heart tissue in the correct spots. This requires accurate definition of the boundaries of the different anatomies. Traditionally, a Clinical Application Specialist (CAS) examines each image frame and draws the boundaries of the existing anatomies.

A model is generated and train to provide an automatic segmentation solution. The model learns to provide a 3D segmentation of the anatomies from a sparse set of frames. In Left atrium ablation, these set of frames are conventionally acquired by inserting the catheter in the right atrium and clocking clockwise and counterclockwise with specific angles to obtain images of the left atrium, left atrial appendage and pulmonary veins. This typical acquisition protocol captures most of the data sets. However, occasionally, the cardiologist may decide to take different views that do not conform to the standard acquisition protocol. Since any model performance degrades when it is tested on a data set drawn from a distribution that is different from the training data set distribution, it becomes crucial to identify such data sets and use them to enrich the training data set. Referring back to FIG. 2, most of the data acquired by the conventional acquisition protocol falls in B2.

For retraining, it is useful to identify data sets in bands B1 and B3 which would correspond to the data sets that deviate from the conventional images whether in the acquisition protocol, patient population, image quality and any other factor that would cause the data set to be underrepresented. To identify such data sets in the way that is impactful clinically, the segmentation outcome is correlated with the final clinical end goal, which is the ablation points.

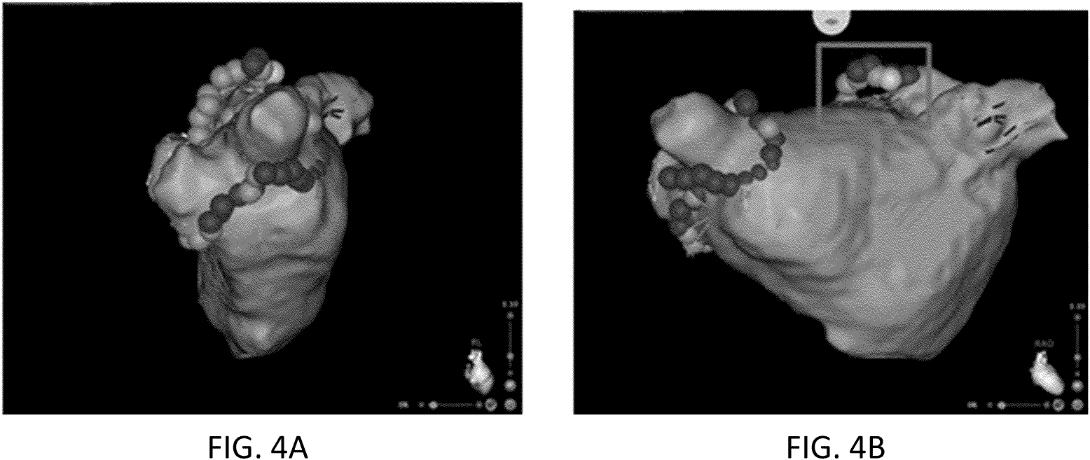

FIGS. 4A and 4B depict an example of the possible location of the ablation tags relative to the segmentation surface. In FIG. 4A, visitags fall perfectly on the segmentation surface. In FIG. 4B, some of the visitags (green box) are floating above the surface, indicating a possibility of under segmentation of the surface. Perfect segmentation means that the ablation points fall perfectly on the segmented surface as depicted in FIG. 4A. If the ablation tags are buried inside the segmentation surface, this would indicate an over segmentation of the boundary since the cardiologist will not ablate in the blood pool. Conversely, ablation tags that are floating above the surface would indicate under segmentation of the boundaries as depicted in FIG. 4B. Since the end goal is to have the ablation points on the segmentation surface, the distance between the ablation tags and the segmentation surface is used as the evaluation metric.

An evaluation metric is computed as follows: Let p be an ablation point that is part of the set of ablation tags P. Let the set V be the set of vertices constituting the segmentation mesh. A mesh is just an example of surface representation although other forms may be used. The surface may be represented in any other form, for example, a 3D binary mask or a cloud of points. ∀p ∈ P, The minimum distance to the segmentation mesh is computed that is represented as:

$${d}_{p} = \text{min} \left|\left|v - p\right|\right| ∀ v ∈ V$$

Then, the metric m is computed as m as m = mean (dₚ) ∀ p ∈ P. The metric m is not restricted to the mean distance error between the ablation tags and the surface. It is used here as an example; however, other metrics can be used. For example, one can use any other statistics such as the maximum or median error or a vector of metrics that contains one or more metrics.

The workflow for enriching the training with the most valuable data sets can be summarized as: 1. Given a data set D0, train a model to generate the automatic segmentation S from a set of sparse frames. 2. Deploy the model in target clinical locations. 3. For every procedure, collect the imaging data and the visitags locations. 4. Compute the Error metric m or the set of metrics. 5. Sort the data sets in a descending order based on the metric m. Recall: the higher the error metric, the more valuable this data set it because the model found it challenging to segment. 6. Choose the top x%, for example 10%, of the sorted data sets, say T0, and add them to the original training data D0 to form the new training data set D1 = D0 ∪ T 0. 7. Retrain the model using the new data set D1. Repeat steps 1-7 with Di = Dᵢ₋₁ ∪ Tᵢ₋₁.

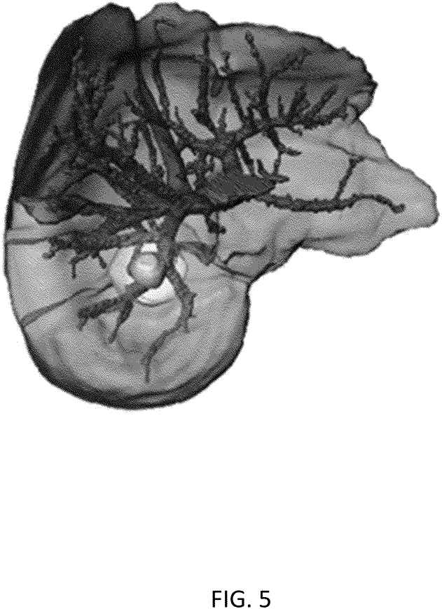

In a second example, a model is used for Liver Resection Surgery Planning from CT scans. Liver resection refers to the process of removing part of the liver that contains tumor(s). The resection subdivides the liver into resected liver and remnant liver. The resection process is not straight forward. It is not a simple removal of the tumor as this might de-vascularize some tissue making it in unviable. The resection process aims at maximizing the remnant tissue, minimizing the resected tissue but taking into consideration that the tumor margin should be maximized and minimizing the de-vascularization. FIG. 5 depicts an example of a liver resection into remnant liver (blue) and resected liver (red) illustrates the idea. The resection surface is the interface between the red segment and the blue segment.

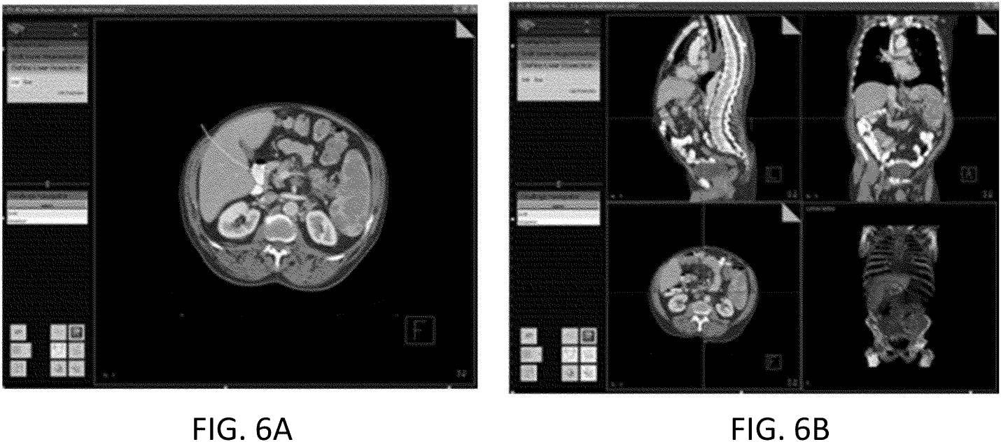

Automatic liver resection surgery planning uses machine learning models to find the optimal resection surface that optimizes the previous factors based on pre-operative CT scans. For example, the clinician would draw one or more contours in the orthogonal planes, and the machine learning algorithm automatically computes the full resection surface in 3D using the minimal user input. FIGS. 6A and 6B depict planning for an automatic liver resection. In FIG. 6A, the user input is shown in yellow, one contour drawing to guide the resection. In FIG. 6B, the results of the automatic resection in 3D (lower right quadrant) with the remnant liver in green and resected liver in red.

In such application, after deployment, the automatic liver resection model is run that provides the suggested resection surface. After the surgery is done, the remnant liver from the resection planning algorithm is registered to the post-operative remnant liver. The evaluation metric is computed to assess how close the automatically computed remnant liver to the actual one. A metric could be the dice coefficient between the two binary masks, the distance between the two surfaces or any other metric that assesses the closeness.

The system for enriching the training with the most valuable data sets can be summarized as: 1) Train the resection algorithm using an initial data set D0. 2. Deploy the resection algorithm in clinical site. 3. For each new case, run the algorithm to compute the remnant liver, R_{ML}. 4. Segment the remnant liver after the surgery, Rₛᵤᵣ. 5. Compute the desired metric or set of metrics, for example DICE (R_{ML},Rₛᵤᵣ). 6. Sort the data sets in an ascending order based on the DICE coefficient. Smaller dice coefficient indicates more challenging data set. 7. Select the top 10% of the sorted data, T, to prioritize in annotation and preprocessing. Add it to the original data set to from a new training set D1 = D0 ∪ T. 9. Retrain the model using the new data set D1. Repeat steps 1-8 with Di = Dᵢ₋₁ U Tᵢ₋₁

At act A150, data sets are identified / selected / flagged that include evaluation metrics outside a predetermined range or that do not meet a threshold level of quality. A group of data sets may be acquired prior to determining which data sets are outside the predetermined range. The range may be determined from the group of data sets. The data sets are that are outside the acceptable range may be data that is an abnormal distance from other the other data. The selected data sets may be taken from the poorly performing data sets, e.g., the most challenging. In an example, only the worst performing / most challenging 5%, 10%, or 20% of data sets may be selected. Different buckets may be used to separate the data sets by how challenging the data sets are. A size or percent may be based on a total number of data sets or how many data sets are appropriate / used during retraining. In an embodiment, an outlier detection component may be used to clean the data sets from the outliers or incorporate modeling of the data probability distribution. Outliers or data sets that are outside so far outside the range may be discarded. These edge cases may be useful in certain applications (and thus may be kept) but may be detrimental to the training process.

In an embodiment, in editing applications, interactive editing algorithms use users’ inputs to correct segmentation. The objective is to be able to perform the editing in the least possible time with the best quality. The evaluation metric may be a number of interactions the radiologist/clinician use to correct the output. Another possible evaluation metric is the number of spots that need editing, or the total time used for editing. These evaluation metrics may be used to prioritize the data sets to add and enrich the training data.

At act A160, the flagged data sets are prioritized during retraining of the machine trained model. Prioritizing may include different actions. In an embodiment, the worst performing data sets are the first to be used to retrain the model as these data sets were the most challenging for the model to predict an outcome for. Referring back to FIG. 2, the model should perform well on the B2 data sets. The B3 datasets may be where the model overperformed. The B1 datasets are where the model underperformed. The B1 and B3 datasets may be flagged to provide diversity for the model with the B1 datasets given priority over the B3 datasets. Depending on the amount of data, processing power, and time frame, only these data sets may be used during a retraining phase. If the capacity exists, additional data sets (for example, from B2) may be used, for example, the next worst performing data sets.

In an embodiment, the flagged data sets are given priority in at least one of data transfer, data anonymization, or preprocessing during retraining of the machine trained model. In this example, when data is collected from one or more clinical sites for use in retraining the model, the data sets that performed the worst (e.g., the worst evaluation metric scores from the clinical site) may be transmitted or processed first. Cleaning the data sets, e.g., preparing the data sets for input into the model for training is a labor and resource intensive task. Each data set must be similar in format, size, information included, etc. For example, certain models may only be able to handle a specific sized image or format. The incoming data sets must be processed to meet these input requirements for each model to be trained. For processing, prioritizing may also include automatically assigning a priority score to the data sets and push them to an annotation queue according to their priority score. In an example, annotation is also a labor-intensive technique, that in some scenarios requires pixel-level accuracy. Annotation may be manually performed and may vary in quality between operators. Prioritizing may include assigning the identified data sets to annotators such that expert annotators get the most difficult data sets to annotate.

In an embodiment, the identified data sets may be used to retrain the model at the clinical site. Based on the availability of computational resources in the deployment sites, online learning can be used to improve the deep learning models on the spot based on the flagged data sets.

In another embodiment, prioritizing includes identifying the most “valuable” datasets to train on at each site in a federated learning setup or site by site. In an example, the evaluation metrics for all data sets from a site (or devices) may be grouped and prioritized group by group. In this way, sites that produce beneficial data may be prioritized over sites that produce redundant data.

The result of the prioritizing is that the data sets that are most useful for the training and retraining of the model may be assured of being used. Redundant data for which the model already performs well on is not prioritized, instead, the redundant data may be used only when time and resources permit.

At act A170, the retrained machine trained model is deployed back to the clinical sites. The steps A110-A170 may be repeated. The smart selection of data using the evaluation metric provides for a reduced cost and time of development and will provides more precise models. Smart selection allows for quicker and more efficient training by selectively identifying the data sets that will impact the performance of the model the most. Smart selection optimizes the development cycle and enables an improved performance of the deployed deep learning models at the lowest possible cost.

FIG. 7 depicts an example of a system 100 for smart selection of training data sets. The system 100 includes a datastore 120, a processor 110, and a model 150 configured to perform a task. The datastore 120 includes a plurality of data sets (data set A, data set B, data set C, etc.) with each data set assigned a priority score based on an evaluation metric that correlates with a clinical outcome. The processor 110 is configured to train the model 150 using the plurality of data sets. The model 150 may be deployed to a clinical site 140. The medical imaging device 130 may generate data on which the model 150 is applied.

The model 150 / neural network (deep learning model or network) may include any architecture or layer structure for deep machine learning. The architecture defines the structure, learnable parameters, and relationships between parameters. In one embodiment, a convolutional or another neural network is used. Any number of layers and nodes within layers may be used. A DenseNet, U-Net, encoder-decoder, Deep Iterative Down-Up CNN, image-to-image and/or another network may be used. Some of the network may include dense blocks (i.e., multiple layers in sequence outputting to the next layer as well as the final layer in the dense block). Any know known or later developed neural network may be used. Any number of hidden layers may be provided between the input layer and output layer.

The datastore 120 / memory is configured to store the network and training data for training or configuration of the network. For example, the configuration, nodes, weights, and other parameters of the machine learned models / networks may be stored in the memory. The datastore 120 may be or include an external storage device, RAM, ROM, database, and/or a local memory (e.g., solid state drive or hard drive). The same or different non-transitory computer readable media may be used for the instructions and other data. The memory may be implemented using a database management system (DBMS) and residing on a memory, such as a hard disk, RAM, or removable media. Alternatively, the memory is internal to the processor (e.g., cache).

The datastore 120 is configured to store a plurality of data sets to be used in training the network. The data sets may be processed or unprocessed. The data sets may be annotated or unannotated. The data sets may include a priority score or evaluation metric that indicates how the processor 110 should prioritize the respective data set for processing or inclusion in the training of the network. In an example, an unprocessed data set with a high priority score / evaluation metric may be pushed to a front of a queue for processing / transmission / annotation etc. An annotated data set with a high priority score / evaluation metric may be pushed to the front of a queue for training the network during a subsequent training phase. The datastore 120 is further configured to store the instructions for performing the methods described herein. The instructions for implementing the processes, methods, and/or techniques discussed herein are provided on non-transitory computer-readable storage media or memories, such as a cache, buffer, RAM, removable media, hard drive, or other computer readable storage media (e.g., the memory). The instructions are executable by the processor 110 or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

The processor 110 is a control processor, image processor, general processor, digital signal processor, three-dimensional data processor, graphics processing unit, application specific integrated circuit, field programmable gate array, artificial intelligence processor, digital circuit, analog circuit, combinations thereof, or other now known or later developed device. The processor 110 is a single device, a plurality of devices, or a network. For more than one device, parallel or sequential division of processing may be used. In one embodiment, the processor is a control processor or other processor of a medical imaging device. The processor 110 operates pursuant to and is configured by stored instructions, hardware, and/or firmware to perform various acts described herein.

The processor 110 is configured to train, configured, test, deploy, and implement the network as described herein. The processor 110 is configured to prioritize data sets based on an associated priority score / evaluation provided by the smart selection process described above. The training by the processor 110 allows the network to learn both the features of the input data and the conversion of those features to the desired output. Backpropagation, RMSprop, ADAM, or another optimization is used in learning the values of the learnable parameters of the network (e.g., the convolutional neural network (CNN) or fully connection network (FCN)). Where the training is supervised, the differences (e.g., L1, L2, mean square error, or other loss) between the estimated output and the ground truth output are minimized.

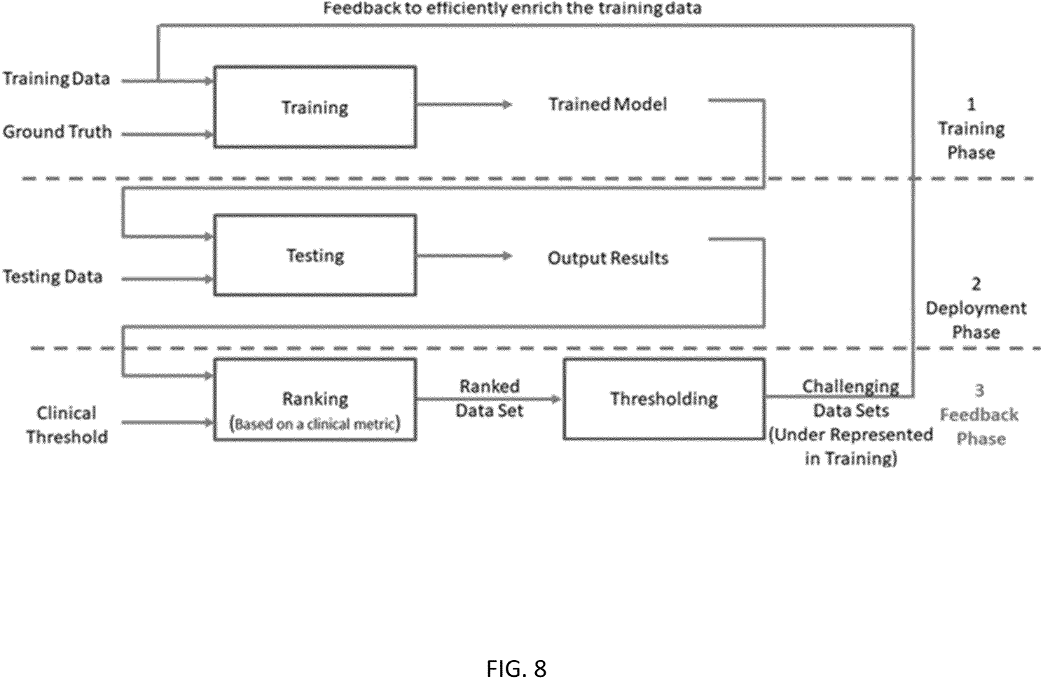

FIG. 8 depicts a workflow for smart selection of training data. Training data is used by the processor 110 for training a model 150. The training data includes ground truth data, for example, stored in the datastore 120. The training phase ends with the output of a trained model 150. The trained model 150 is deployed to a clinical site 140 during the deployment phase. Testing data / clinical data is input into the trained model 150 which outputs results. The output results and a clinical evaluation metric are used to rank the data sets. Challenging data sets are prioritized to efficiently enrich the training data for a subsequent round of training.

The display 115 is configured to display or otherwise provide information to a user. The display is a CRT, LCD, projector, plasma, printer, tablet, smart phone or other now known or later developed display device for displaying the output.

The clinical site 140 may connect to the system 100 via a network. The network is a local area, wide area, enterprise, another network, or combinations thereof. In one embodiment, the network is, at least in part, the Internet. Using TCP/IP communications, the network provides for communication between the clinical site 140 and the system 100. Any format for communications may be used. In other embodiments, dedicated or direct communication is used. The clinical site 140 may include a server, for example, a processor or group of processors. More than one server may be provided. The server is configured by hardware and/or software. The server may be configured to handle a portion of the processing of the clinical data or training data. The server may be configured to train the networks or models using labeled or unlabeled datasets.

The medical imaging device 130 may include any medical imaging device 130 that is configured to acquire medical imaging data of a patient or object. The medical imaging data may be input into a trained model 150 that is stored at the clinical site 140 or elsewhere. The output of the trained model may provide data sets for future training of the model. The medical imaging device 130 may also acquire information or data that is used to compute an evaluation metric or priority score that is used for smart selection of data sets for training of the model.

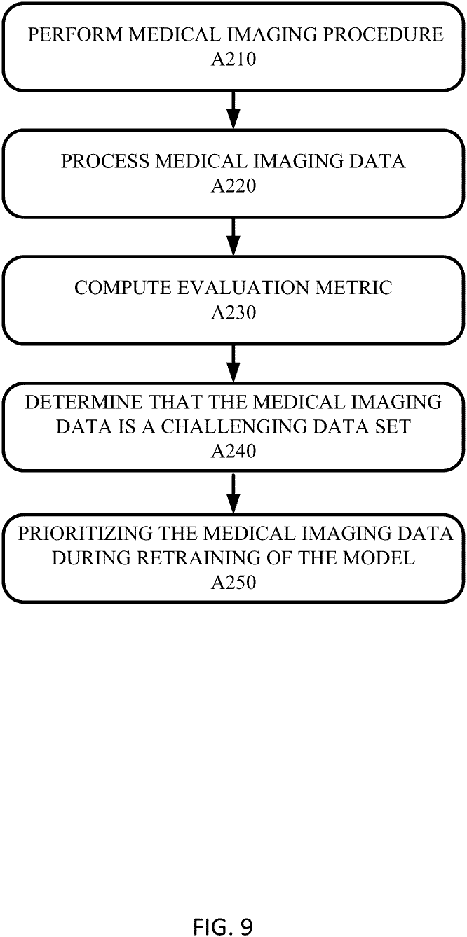

FIG. 9 depicts an example method for smart selection of data sets for training a model. As presented in the following sections, the acts may be performed using any combination of the components indicated in FIG. 7. The following acts may be performed by the system 100, processor 110, datastore 120, clinical site 140, medical imaging device 130, or a combination thereof. Additional, different, or fewer acts may be provided. The acts are performed in the order shown or other orders. The acts may also be repeated. Certain acts may be skipped.

At Act A210, the medical imaging device 130 performs a medical imaging procedure to acquire medical imaging data. At Act A220, the medical imaging data is processed by a machine learned model 150 to generate a data set. At Act A230, an evaluation metric is computed that correlated with an outcome of a medical procedure or diagnosis related to the medical imaging procedure. At Act A240, a determination is made based on the evaluation metric that the data set is a challenging data set. At Act A250, the challenging data set is prioritized during a subsequent round of training of the model 150.

It is to be understood that the elements and features recited in the appended claims may be combined in different ways to produce new claims that likewise fall within the scope of the present invention. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description. Independent of the grammatical term usage, individuals with male, female or other gender identities are included within the term.

1. A method for smart selection of training data sets, the method comprising:
machine training a model to perform a task;
deploying the machine trained model to a clinical environment;
computing an evaluation metric that correlates with a clinical outcome for each instance of the machine trained model performing the task for a medical procedure; and
flagging data sets that are challenging for the machine trained model based on the evaluation metrics.

2. The method of claim 1, further comprising:
prioritizing the flagged data sets during retraining of the machine trained model.

3. The method of claim 2, wherein prioritizing the flagged data sets comprises wherein the flagged data sets are given priority in at least one of data transfer, data anonymization, or preprocessing during retraining of the machine trained model.

4. The method of claim 2, wherein flagging the data sets comprises automatically assigning a priority score to the data sets and prioritizing the data sets comprises pushing the data sets to an annotation queue according to their priority score.

5. The method of claim 2, wherein prioritizing the flagged data sets comprises assigning the flagged data sets to annotators such that expert annotators get the most difficult data sets to annotate.

6. The method of claim 2, wherein prioritizing comprises identifying sites in a federated learning setup that include the most challenging data sets.

7. The method of claim 1, wherein during machine training of the model, the model is tested using a testing metric that is different than the evaluation metric.

8. The method of claim 1, wherein the task comprises image segmentation of medical imaging data acquired using one of MRI, CT, X-ray, or Ultrasound.

9. The method of claim 1, wherein the evaluation metric comprises an assessment of how well an output of the machine trained model guided a clinician during the medical procedure subsequent to the performance of the task.

10. The method of claim 1, wherein flagging comprises flagging data sets that are the ten percent most challenging data sets.

11. A system for smart data selection for training a deep learning network, comprising:
a datastore configured to store a plurality of data sets, wherein each data set of the plurality of data sets is assigned an evaluation metric that correlates with a clinical outcome associated with a respective data set;
the deep learning network configured to perform a task; and
a processor configured to train the deep learning network using the plurality of data sets, the processor configured to prioritize data sets for use in the training based on the evaluation metric.

12. The system of claim 11, wherein during training of the deep learning network, the deep learning network is tested using a testing metric that is different than the evaluation metric.

13. The system of claim 11, wherein the evaluation metric comprises an assessment of how well an output of the deep learning network guided a clinician during a medical procedure.

14. The system of claim 11, wherein the processor is configured to prioritize the data sets in at least one of data transfer, data anonymization, or preprocessing during training of the deep learning network.

15. The system of claim 11, wherein the processor is configured to prioritize the data sets by assigning the data sets to annotators such that expert annotators get the most difficult data sets to annotate.

16. The system of claim 11, wherein the processor is configured to assign the data sets a priority score based on the evaluation metric and prioritize the data sets by pushing the data sets to an annotation queue according to their priority score.

17. The system of claim 11, wherein the processor is configured to only use the ten percent most challenging data sets based on the evaluation metric for subsequent training of the deep learning network.

18. A method for smart data collection, the method comprising:
performing a medical imaging procedure to generate medical imaging data;
processing the medical imaging data using a machine learned network;
computing an evaluation metric for the processed medical imaging data based on a clinical outcome related to the medical imaging procedure;
determining, based on the evaluation metric, that the medical imaging data comprises a challenging data set; and
prioritizing the medical imaging data for retraining of the machine learned network.

19. The method of claim 18, wherein prioritizing comprises at least one of data transfer, data anonymization, or preprocessing of the medical imaging data during retraining of the machine learned network.

20. The method of claim 18, wherein prioritizing the medical imaging data comprises assigning the medical imaging data to annotators such that an expert annotator gets the medical imaging data to annotate.
